(19) United States
(12) Patent Application Publication
(10) Pub. No.: US 2019/0143379 A1
(43) Pub. Date: May 16, 2019

(54) CLEANING SYSTEM AND CLEANING METHOD FOR A STYLUS OF AN INOCULATION APPARATUS

(71) Applicant: Don Whitley Scientific Limited, West Yorkshire (GB)

(72) Inventors: Evan Jonathan Kitsell, West Yorkshire (GB); David Boast, West Yorkshire (GB)

(73) Assignee: Don Whitley Scientific Limited, West Yorkshire (GB)

(21) Appl. No.: 16/099,426

(22) PCT Filed: May 8, 2017
(86) PCT No.: PCT/GB2017/051274

§ 371 Date: Nov. 6, 2018

(30) Foreign Application Priority Data
May 8, 2016 (GB) 1608009.5

(51) Int. Cl. B08B 3/04; C12M 1/26; G01N 35/10

(52) U.S. Cl. CPC B08B 3/04; G01N 35/1004; C12M 33/00

(57) ABSTRACT

A cleaning system for cleaning a stylus of an inoculation apparatus comprises a bath with a first washing trough to hold a first washing fluid and a first weir defining a first washing fluid level, a second washing trough to hold a second washing fluid and a second weir defining a second washing fluid level higher than the first washing fluid level so the stylus is immersible to a greater depth in the second washing fluid than in the first washing fluid. A third washing trough may hold a third washing fluid with a third weir defining a third washing fluid level higher than the first washing fluid level so the stylus is immersible to a greater depth in the third washing fluid than in the first washing fluid. An inoculation system may comprise the cleaning system and may employ an automated method of cleaning the stylus.

### FIELD OF THE INVENTION

The present invention relates to a cleaning system and cleaning method for a stylus of an inoculation apparatus.

### BACKGROUND OF THE INVENTION

During microbiological testing an inoculation apparatus is commonly used to deposit a test sample on a substrate. The inoculation apparatus typically deposits the test sample on the substrate in a predetermined pattern, such as a spiral pattern. As a result, an inoculation apparatus is sometimes referred to as a spiral plater.

An inoculating apparatus conventionally uses a stylus to collect and deposit the test sample on the substrate. To prevent contamination between test samples, the stylus is cleaned after each deposition.

One known way of cleaning the stylus involves dipping the stylus in a washing fluid. However, it has been found that this one-step washing process may not sufficiently clean the stylus. Moreover, any washing fluid remaining on the surface of the stylus may be transferred to next test sample. Conventional cleaning processes also require an operator to regularly top-up the washing fluid. However, manual replenishing is not reliable and human-error may lead to an undersupply or oversupply of washing fluid.

### SUMMARY OF INVENTION

The present invention seeks to address and/or ameliorate the one or more problems described above. The present invention seeks to provide an improved cleaning system and cleaning method for a stylus of an inoculation apparatus.

A first aspect of invention relates to a cleaning system for cleaning a stylus of an inoculation apparatus.

The stylus of the inoculation system may be any suitable device for depositing a test sample on a substrate. The stylus may, for example, comprise a syringe, pipette, nozzle or loop.

The cleaning system comprises a bath with a plurality of washing troughs in which a stylus can be immersed in washing fluid. As a result, the bath provides multiple and sequential washing steps.

Each washing trough comprises a weir configured to define a washing fluid level and thereby automatically control the level of washing fluid in the washing trough. As a result, the washing fluid in each washing trough has a predetermined maximum level and cannot be exceeded. By using weirs to define fluid levels in the washing troughs, the volume of washing fluid required to fill each trough to the requisite fluid level is known and no complex regulation or measuring of washing fluid is required. The weirs of each washing trough are configured such that, when each washing trough is filled with washing fluid to the predetermined washing fluid level, the level of washing fluid in each washing trough is sufficient to allow the stylus to be immersed in the washing fluid to a desired depth so as to achieve a desired washing effect.

The bath may comprise:
a first washing trough with a first weir configured to define a predetermined first washing fluid level; and
a second washing trough with a second weir configured to define a predetermined second washing fluid level, wherein the second washing fluid level is higher than the first washing fluid level.

Thus, during use, when the washing troughs are filled with washing fluid to the predetermined fluid levels, the second washing trough has a higher level of washing fluid than the first washing trough. By providing the higher level of washing fluid in the second washing trough to the first washing trough, the stylus is thereby immersible to a greater depth in the second washing fluid than in the first washing fluid. Immersing the stylus to a greater depth in the second washing fluid, advantageously enhances the washing of the stylus and reduces the risk of the first washing fluid remaining on the stylus. For example, if the washing fluid in the first trough comprises a sanitizer and the washing fluid in the second trough comprises a rinser then, by immersing the stylus to a greater depth in the rinser than the sanitizer, the rinsing of the stylus is improved and the transfer of residual sanitizer is limited.

To further enhance the washing of the stylus, the bath may comprise a third washing trough with a third weir configured to define a predetermined third washing fluid level, wherein the third washing fluid level is higher than the first fluid level. It follows that the stylus is thereby immersible to a greater depth in the third washing fluid than in the first washing fluid. The third washing fluid may, for example, comprise a rinser to aid the rinsing of the stylus following sanitizing in the first washing trough.

The washing troughs may have any suitable shape and size to hold washing fluid. The washing troughs may have a generally cuboid shape in the form of narrow, elongate recesses. As a result, the surface area of the washing fluids is limited and so the evaporation of the washing fluids is limited. To help provide a compact bath design, the washing troughs are preferably arranged in parallel. To further reduce the size of the bath, the washing troughs may be integrally formed as part of a bath body. Alternatively, the washing troughs may be separately formed. The separately formed washing troughs may comprise coupling means configured to couple the washing troughs together in a sequential arrangement.

The bath may be mountable on a working platform of the inoculation apparatus. The bath may be mountable on the working platform adjacent or convenient to a substrate table so as to limit the movement of the stylus between a substrate and the bath after depositing a test sample on the substrate. Preferably, the bath may be removably mountable on the inoculation apparatus to allow for the removal, cleaning, refitting or replacement of the bath. The narrow configuration of the bath may allow for the length of the working platform to be shortened, and thereby the overall size of the inoculation apparatus reduced.

The cleaning system may comprise drive means to move the stylus sequentially between the washing troughs so as to allow the stylus to be initially washed in the first washing trough and subsequently washed in the second washing trough, and optionally washed in the third washing trough. To regulate the position of the stylus in each trough, the drive means may be configured to insert the stylus in each trough to a predetermined distance from the bottom of each trough. The drive means may comprise an arm configured to translate and/or rotate the stylus to a desired position.

The cleaning system may comprise washing fluid supply means to supply washing fluid to the troughs of the bath. The supply means may be configured to prime the bath so as to fill each trough with washing fluid to the predetermined fluid levels. To replace any washing fluid consumed during the washing process, the supply means may also be configured to refill each trough with washing fluid to the predetermined fluid levels by adding a replenishing volume of washing fluid. To help limit the contamination of washing fluid in each washing trough, refresh and maintain the efficacy of the washing fluid, the replenishing volume of washing fluid added to each washing trough is preferably greater than the volume of washing fluid consumed during the wash cycle. Advantageously, since the volume of washing fluid consumed during the wash cycle is known and the requisite fluid level in each trough is pre-set by the weirs the replenishing volume of washing flushing can be predetermined.

The washing fluid supply means may comprise a reserve of washing fluid and a spout in fluid communication with the reserve. To supply each trough separately, the washing fluid supply means may comprise:
a reserve of first washing fluid, a first supply pump and a first spout to direct first washing fluid into the first washing trough; and
a reserve of second washing fluid, a second supply pump and a second spout to direct second washing fluid into the second washing trough.

If the bath comprises a third washing trough, the washing fluid means may comprise a reserve of third washing fluid, a third supply pump and a third spout to direct third fluid into the third washing trough.

The first washing fluid preferably comprises a sanitizer to sterilise the stylus. The sanitizer may comprise any suitable sanitizer, for example an alcohol or a chlorine-based disinfectant. The first washing fluid may additionally comprise a detergent to aid the cleaning of the stylus and removal of test sample from the stylus.

The second washing fluid preferably comprises a rinser to rinse the stylus following the sterilizing of the stylus by the first washing fluid. The rinser may comprise any suitable rinser, for example deionised water or sterilised water. The second washing fluid may further comprise a detergent to aid the removal of test sample and sanitizer from the stylus.

If the bath comprises a third washing trough, the third washing fluid preferably comprises a rinser to repeat rinse the stylus. The third washing fluid may comprise any suitable rinser and the third washing fluid and second washing fluid may comprise the same rinser. The third washing fluid may comprise a detergent.

The spouts may be configured in spaced relation to the respective washing troughs. For example, the spouts may be arranged above the respective troughs by a predetermined height.

By forming the non-continuous path, the back flow of washing fluid from the washing troughs and thereby contamination of the spouts is restricted.

The cleaning system may comprise an overflow trough configured to collect any excess washing fluid overflowing from the washing troughs. The overflow trough is preferably arranged adjacent to weirs of the washing troughs to receive any excess washing fluid overflowing the weirs. Washing fluid may overflow the weirs into the overflow trough during the filling of the troughs, immersion of stylus and/or replenishing of washing fluid.

The overflow trough may be integrally formed with the washing troughs of the bath. Alternatively, the overflow trough may be separately formed. The separately formed overflow trough may comprise coupling means configured to couple the overflow trough to at least one washing trough.

The cleaning system may comprise drain means to drain the overflow trough. The draining means may comprise a drain vessel in fluid communication with the overflow trough. The drain vessel may be configured to allow the overflow fluid to drain from the overflow trough to the drain vessel under gravity. Optionally, the drain means may comprise a draining pump to withdraw overflow fluid from the overflow trough into the drain vessel when desired.

If the stylus comprises an internal bore and/or chamber then internal surfaces of the stylus will require washing to restrict contamination. Hence, the cleaning system may comprise flushing means to flush through the stylus with washing fluid. The flushing means is configured to flush the stylus when the stylus is immersed in the washing fluid of at least one of the washing troughs and by drawing a volume of the washing fluid through the stylus. The flushing means comprises a flushing vessel in fluid communication with the stylus and a flushing vacuum pump to draw a predetermined volume of washing fluid, from the washing fluid in which the stylus is immersed, through the stylus and into the flushing vessel. The stylus is preferably flushed with each washing fluid as the stylus moves sequentially from trough to trough so that the washing of the internal surfaces of the stylus is consistent with the washing of the external surfaces of the stylus. Advantageously, the gap spaced between the bottom of the trough and the immersed stylus allows for particulates to remain settled at the bottom of trough during the flushing process.

The cleaning system may comprise control means to automate the cleaning process. The control means may be configured to control the operation of the drive means so as to automate the moving of the stylus during the washing process. The control means may be configured to control the operation of the washing fluid supply means so as to automate the filling and/or replenishing of washing fluid in each washing trough. For example, the control means may automatically activate the replenishing the washing fluid in each trough after identifying the drive means has withdrawn the stylus from the last washing trough. The control means may be configured to control the operation of the drain means to automate the draining of the overflow trough. The control means may be configured to operate the flushing means to automate the flushing process. The control means may comprise an operator device to allow for operator input in the operation of the cleaning system and/or operator warnings.

The cleaning system may comprise a cover arrangeable to protect the bath from airborne contaminants. As a result, the efficacy of the washing fluid is enhanced. The cover may comprises a canopy spaced from the bath by a predetermined distance and shaped to restrict air flow to the bath. For example, the cover may comprise a canopy arranged a predetermined distance above the bath and configured to guide air flow away from the bath. If the bath is mountable on the workstation platform of the inoculation device then the cover may be configured to extend at least substantially along the length of the workstation platform.

A second aspect of the invention relates to an automated method of cleaning a stylus of an inoculation apparatus comprising:
filling a first washing trough with a first washing fluid to a first fluid level defined by a first weir;
filling a second washing trough with a second washing fluid to a second fluid level defined by a second weir, wherein the second fluid level is higher than the first fluid level; and optionally
filling a third washing trough with a third washing fluid to a third fluid level defined by a third weir, wherein the third fluid level is higher than the first fluid level.

The method further comprising:
immersing a stylus at a first depth in a first washing fluid of the first washing trough;
withdrawing the stylus from the first washing fluid of the first washing trough and immersing a stylus at a second depth in a second washing fluid of the second washing trough, wherein the second depth is greater than the first depth; and optionally
withdrawing the stylus from the second washing fluid of the second washing trough and immersing the stylus at a third depth in a third washing fluid of the third washing trough, wherein the third depth is greater than the first depth.

The method further comprising:
when the stylus is immersed in the first washing fluid of the first washing trough, drawing a predetermined flushing volume of first washing fluid through the stylus; and/or
when the stylus is immersed in the second washing fluid of the second washing trough, drawing a predetermined flushing volume of second washing fluid through the stylus; and/or optionally
when the stylus is immersed in the third washing fluid of the third washing trough, drawing a predetermined flushing volume of third washing fluid through the stylus

The method further comprising
refilling the first washing trough to the first fluid level with a predetermined replenishing volume of first washing fluid, wherein the replenishing volume is greater than the first washing fluid consumed during washing in the first washing trough;
refilling the second washing trough to the second fluid level with a predetermined replenishing volume of second washing fluid, wherein the replenishing volume is greater than the washing fluid consumed during the washing in the second washing trough; and optionally
refilling the third washing trough to the third fluid level with a predetermined replenishing volume of third washing fluid, wherein the replenishing volume is greater than the washing fluid consumed during the washing in the third washing trough.

A third aspect of the invention relates to an inoculation apparatus comprising:
a stylus for depositing a test sample on a substrate;
a cleaning system for cleaning the stylus after each deposition according to the first aspect of the invention.

### BRIEF DESCRIPTION OF THE DRAWINGS

For a better understanding of the present invention and to show how it may be carried into effect, reference will now be made, by way of example only, to the accompanying drawings, in which:

FIG. 1 is a perspective view of an embodiment of a bath of a cleaning system according to the present invention;

FIG. 2 is a cross-sectional view showing the weirs of the embodiment of the bath of FIG. 1;

FIG. 3 is a cross-sectional view showing the different washing fluid levels of the embodiment of the bath of FIG. 1;

FIG. 4 is a cross-sectional view showing how a stylus can be immersed in the different depths of washing fluid level in the embodiment of the bath of FIG. 1;

FIG. 5 is a schematic, side view of the embodiment of the bath of FIG. 1, washing fluid supply means, drain means and control means of an embodiment of the cleaning system according to the present invention; and

FIG. 6 is a top view showing the embodiment of the bath of FIG. 1 mounted on a working platform of an inoculation apparatus according to the present invention.

### DETAILED DESCRIPTION OF THE EMBODIMENT OF THE INVENTION

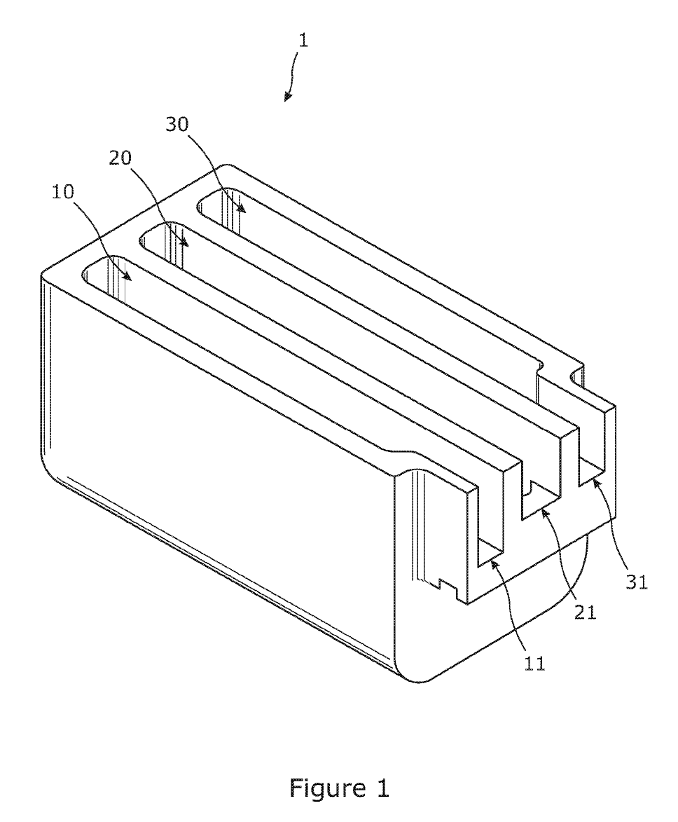

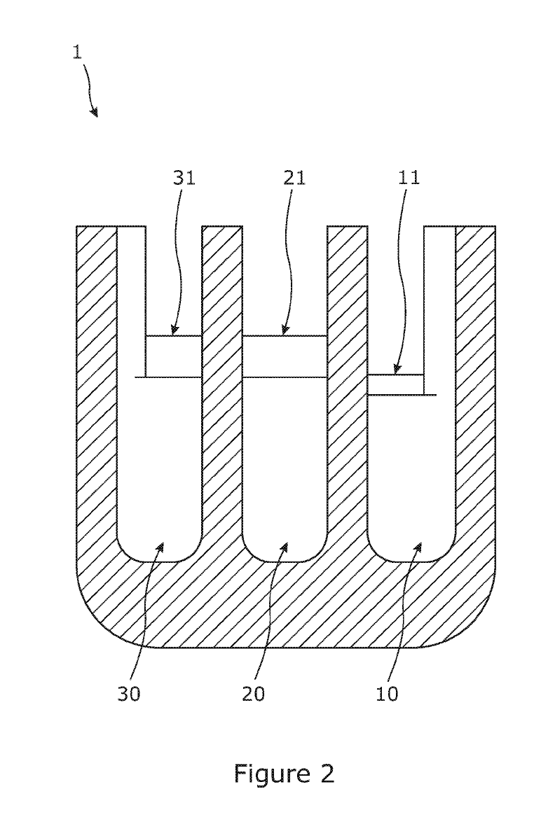

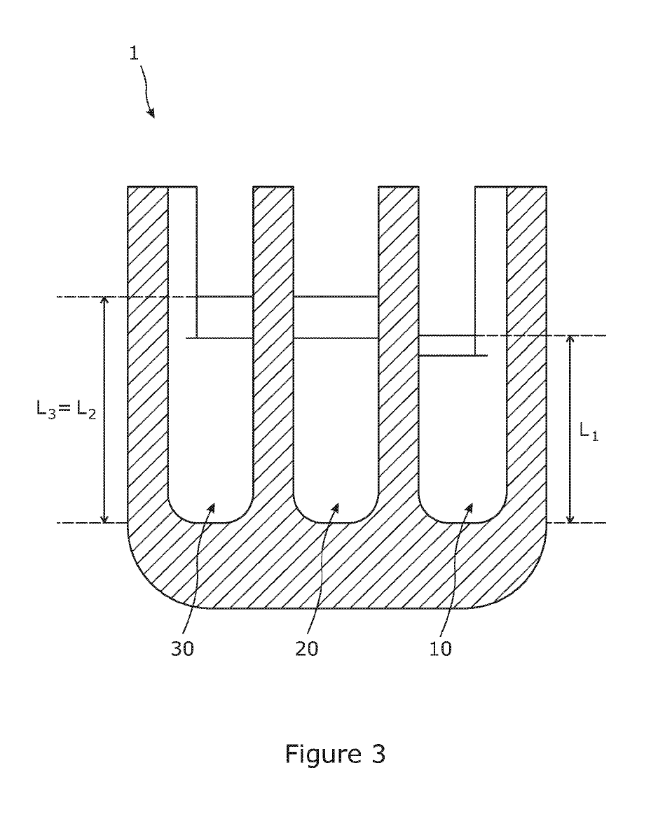

FIGS. 1 to 6 depict an embodiment of a cleaning system for cleaning a stylus (S) in an inoculation system according to the present invention.

The cleaning system comprises a bath (1) with a plurality of washing troughs configured to hold washing fluid. As depicted in FIGS. 1 to 6, this particular embodiment of the bath (1) comprises three washing troughs—a first washing trough (10) for holding a disinfectant, a second washing trough (20) for holding a first rinser and a third washing trough (30) for holding a second rinser.

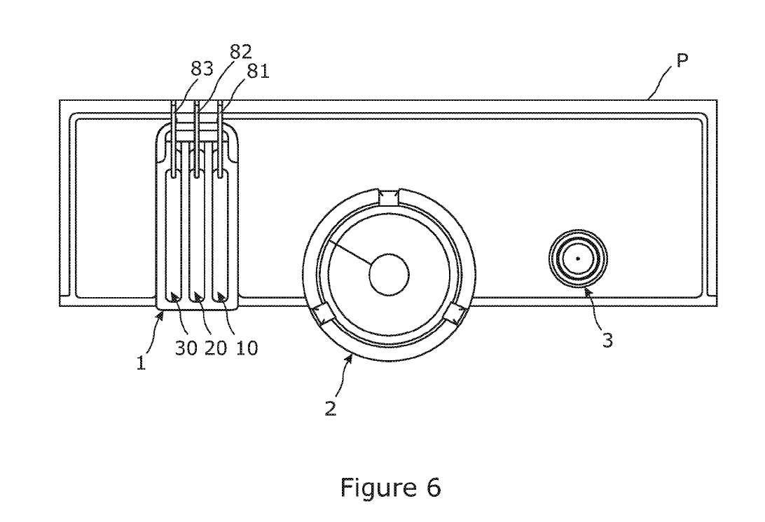

The three washing troughs are cuboid-shaped recesses, integrally formed in the body of the bath. Each trough is approximately 80 mm long, approximately 10 mm wide and approximately 40 mm deep. The troughs are aligned in parallel to allow for sequential access by the stylus. The narrow shape of the troughs helpfully limits the washing fluid surface area so as to restrict the evaporation of the washing fluids. The bath has a compact design due to the configuration of the washing troughs and is approximately 115 mm long, approximately 50 mm wide and approximately 45 mm. FIG. 6 shows the bath (1) mounted in-situ on the working platform (P) of an inoculation apparatus. The bath is mounted on the working platform (P) so that the longitudinal axis of each trough extends generally perpendicular to the direction of movement of the stylus between the troughs. Due to the miniaturisation of the bath, the length of the working platform, and thereby the overall size of the inoculation apparatus, is reduced. The bath (1) is arranged adjacent to a substrate table (2) so as to minimise the movement of the stylus (S) after deposition between a substrate and the bath. To avoid a direct route of transmission between the bath and the sample, a sample supply (3) is arranged on the opposing side of the substrate table (2) to the bath. The bath is removably mounted on the working platform to allow the bath to be removed, refitted or replaced.

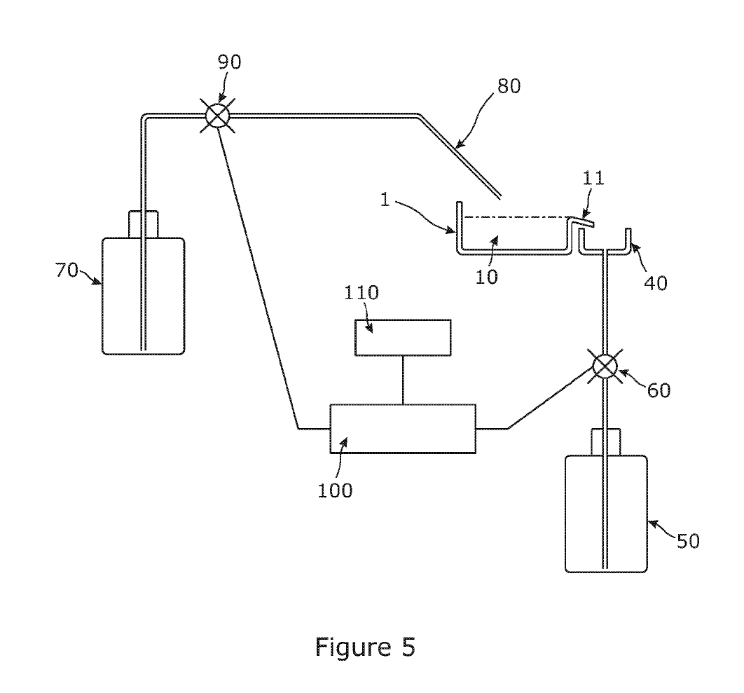

The cleaning system comprises washing fluid supply means to supply the washing fluids to the washing troughs of the bath. As shown in FIGS. 5 and 6, the supply means comprises a reserve of disinfectant (70), a first spout (80/81) to direct disinfectant into the first washing trough (10) and a first supply pump (90) to pump disinfectant from the reserve to the first spout; a reserve of first rinser (not shown), a second spout (82) to direct first rinser into the second washing trough (20) and a second supply pump (not shown); a reserve of second rinser (not shown), a third spout (83) to direct the second rinser into the third washing trough and a third supply pump (not shown). The spouts are spaced from the washing troughs to avoid any backflow of washing fluid. The supply means is configured to prime the bath prior to the washing process so to as to fill each troughs with the requisite washing fluid.

As shown in FIGS. 1 to 3, each washing trough comprises a weir to automatically control the level of washing fluid that can be held in each trough. The weirs are formed in the rear walls of the washing troughs. In the embodiment depicted, the weirs of the second trough and third trough are configured to define the same washing fluid level (L2=L3) and this washing fluid level is greater than the washing fluid level for the first trough (L2=L3>L1). The first washing trough (10) comprises a first weir (11) to limit the level of disinfectant that can be held in the first washing trough to approximately 20 mm (L1). The second washing trough (20) comprises a second weir (21) to limit the level of first rinser that can be held in the second washing trough to approximately 25 mm (L2). The third washing trough (30) comprises a third weir (31) to limit the level of second rinser that can be held in the third washing trough to approximately 25 mm (L3).

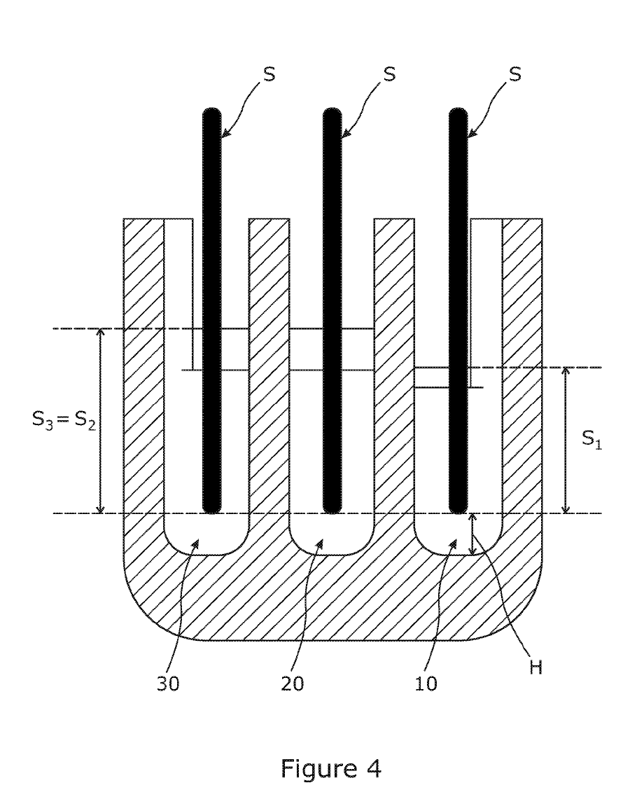

The cleaning system comprises a drive means (not shown) to sequentially immerse the stylus in each of the three washing fluids during the washing process. Due to the parallel alignment of the troughs and arrangement of the bath on the inoculation apparatus, the drive means in this embodiment is configured to slide the stylus along a horizontal axis so as to move the stylus sequentially from the first trough to the third trough. The drive means is also configured to raise/lower the stylus along a vertical axis so as to immerse/withdraw the stylus in the washing fluid held in each trough. As can be seen from FIG. 4, the drive means is configured to immerse the stylus in the washing liquids by lowering the stylus (S) into each trough to the same predetermined height (H) above the bottom of each trough. Due to the weirs defining different fluid levels in the troughs, the stylus (S) is immersed to a depth of approximately 21 mm (S2=S3) in both the first rinser (S2) and the second rinser (S3) and the stylus is immersed to a depth of approximately 16 mm (S1) in the disinfectant. As a result, the stylus is immersed to a greater depth in the first rinser and second rinser than the disinfectant. This rinsing action improves the cleaning of the stylus and improves the removal of any residual disinfectant from the stylus.

FIG. 5 shows that the embodiment of the cleaning system comprises an overflow trough (40) that is separately formed from the trough and that is arranged adjacent to the weirs of the troughs so as to collect overflowing washing fluid. The overflow trough is drained into a drain vessel (50), when desired, using a drain pump (60).

In this embodiment, the stylus is a suction-type stylus for sucking a volume of test sample from the test sample supply (3) into the stylus prior to deposition on a substrate. To clean the internal surfaces of the stylus, the cleaning means comprises flushing means to flush through the stylus with the disinfectant, first rinser and second rinser. The flushing means comprises a flushing pump (not shown) to draw a predetermined volume of disinfectant, first rinser and second rinser through the stylus and into a flush vessel (not shown), when the stylus is sequentially immersed in washing fluid of the washing troughs.

Since washing fluid is consumed during the washing process, the supply means is also configured to replenish washing fluid to the fluid levels defined by the weirs in each trough.

Since the volume of the washing fluid consumed during the washing process is predetermined, the supply means is configured to supply a predetermined replenishing volume of washing fluid to each trough. Preferably, the predetermined replenishing volume is greater than the consumed volume so as to generate an overflow to help refresh and remove any floating contaminates in the washing fluid.

In the embodiment depicted in FIG. 5, the cleaning system comprises control means 100 to automate the operation of the cleaning system. The control means is configured to control the drive means so as to automate the movement of the stylus between the washing troughs, to control the washing fluid supply means so as to automate the supply of washing fluid, to control the drain means so to as to automate the draining of the overflow trough and to control the flushing means so as to automate the flushing of washing fluid through the stylus. The control means preferably comprises an operator device 110 to allow for operator input and/or provide operator warnings.

In the embodiment, the cleaning system comprises a canopy (not shown) to guide air flow and any airborne contaminants away from the bath (1), substrate table (2) and sample supply (3) on the working platform (P).

Although the invention has been described above with reference to one or more preferred embodiments, it will be appreciated that various changes or modifications may be made without departing from the scope of the invention as defined in the appended claims.

1. A cleaning system for cleaning a stylus of an inoculation apparatus comprising a bath, the bath comprising:
a first washing trough to hold a first washing fluid and comprising a first weir defining a first washing fluid level;
a second washing trough to hold a second washing fluid and comprising a second weir defining a second washing fluid level, wherein the second washing fluid level is higher than the first washing fluid level and so the stylus is thereby immersible to a greater depth in the second washing fluid than in the first washing fluid; and/or
a third washing trough to hold a third washing fluid and comprising a third weir defining a third washing fluid level, wherein the third washing fluid level is higher than the first washing fluid level and so the stylus is thereby immersible to a greater depth in the third washing fluid than in first washing fluid.

2. The cleaning system of claim 1, wherein the troughs are integrally formed or separately formed.

3. The cleaning system of claim 1, wherein the bath is configured to be removably mountable on the inoculation apparatus, preferably on a workstation platform of the inoculation apparatus.

4. The cleaning system of claim 1, further comprising a drive means configured to sequentially immerse the stylus in the first washing fluid of the first washing trough and subsequently the second washing fluid of the second washing trough, and/or the third washing fluid of the third washing trough.

5. The cleaning system of claim 4, wherein the drive means is configured to immerse the stylus by inserting the stylus in the said washing trough, a predetermined distance from the bottom the said washing trough.

6. The cleaning system of claim 1, further comprising a flushing means to flush washing fluid through the stylus when the stylus is immersed in the washing fluid of at least one washing trough, the flushing means comprising:
a flushing vessel in fluid communication with the stylus; and
a flushing pump configured to draw a predetermined flushing volume of washing fluid through the stylus to the flushing vessel.

7. The cleaning system of claim 1, further comprising supply means configured to supply washing fluid to each washing trough, the supply means configured:
to fill each washing trough with washing fluid to the respective washing fluid levels prior to immersing the stylus; and
to replenish the washing fluid in each washing trough to the respective washing fluid levels following the immersion and withdrawal of the stylus from the washing fluid.

8. The cleaning system of claim 7, wherein the supply means is configured to replenish the washing fluid in each washing trough by adding a predetermined replenishing volume of washing fluid to each washing trough, wherein the replenishing volume is greater than the volume of washing fluid consumed from each respective trough during the wash cycle.

9. The cleaning system of claim 7, wherein the supply means comprises:
a first reserve of first washing fluid, a first pump and a first spout in fluid communication with the first reserve and arranged in spaced relation above the first washing trough;
a second reserve of second washing fluid, a second pump and a second spout in fluid communication with the second washing fluid and arranged in spaced relation above the second washing trough; and/or
a reserve of third washing fluid, a third pump and a third spout arranged in spaced relation above the third washing trough.

10. The cleaning system of claim 1, wherein the first washing fluid comprises a sanitizer, the second washing fluid comprises a rinser and/or the third washing fluid comprises a rinser.

11. The cleaning system of claim 1, further comprising an overflow trough arranged adjacent to the weirs of the washing troughs.

12. The cleaning system of claim 11, further comprising a drain means comprising a drain vessel, and/or a drain pump to withdraw fluid from the overflow trough and into the drain vessel.

13. The cleaning system of claim 1, further comprising control means to automatically control the cleaning system.

14. The cleaning system of claim 13, wherein the control means comprises an operator device comprising operator input and/or operator warning.

15. The cleaning system of claim 1, further comprising a cover to guide airflow away from the bath and thereby protect the bath from airborne contamination.

16. An inoculation apparatus comprising a cleaning system as defined in claim 1.

17. An automated method for cleaning a stylus of an inoculation apparatus comprising:
filling a first washing trough with a first washing fluid to a first washing fluid level defined by a first weir;
filling a second washing trough with a second washing fluid to a second washing fluid level defined by a second weir, wherein the second fluid level is higher than the first fluid level; and/or
filling a third washing trough with a third washing fluid to a third fluid level defined by a third weir, wherein the third fluid level is higher than the first fluid level.

18. The method of claim 17, further comprising:
immersing a stylus at a first depth in the first washing fluid of the first washing trough;
withdrawing the stylus from the first washing fluid of the first washing trough and immersing a stylus at a second depth in the second washing fluid of the second washing trough, wherein the second depth is greater than the first depth; and/or
withdrawing the stylus from the second washing fluid of the second washing trough and immersing the stylus at a third depth in the third washing fluid of the third washing trough, wherein the third depth is greater than the first depth.

19. The method of claim 18, further comprising:
when the stylus is immersed in the first washing fluid of the first washing trough, drawing a predetermined flushing volume of first washing fluid through the stylus; and/or
when the stylus is immersed in the second washing fluid of the second washing trough, drawing a predetermined flushing volume of second washing fluid through the stylus; and/or
when the stylus is immersed in the third washing fluid of the third washing trough, drawing a predetermined flushing volume of third washing fluid through the stylus.

20. The method of claim 18, further comprising:
refilling the first washing trough to the first washing fluid level with a predetermined replenishing volume of first washing fluid, wherein the replenishing volume is greater than the volume of first washing fluid consumed during washing in the first washing trough;
refilling the second washing trough to the second washing fluid level with a predetermined replenishing volume of second washing fluid, wherein the replenishing volume is greater than the volume of washing fluid consumed during the washing in the second washing trough; and/or
refilling the third washing trough to the third fluid level with a predetermined replenishing volume of third washing fluid, wherein the replenishing volume is greater than the volume of washing fluid consumed during the washing in the third washing trough.
